(19) United States
(12) Patent Application Publication
(10) Pub. No.: US 2005/0090453 A1
(43) Pub. Date: Apr. 28, 2005

(54) Methods of modulating IL-6

(75) Inventors: Stephen John Carter, Woodvale (AU); Richard Lake, Mount Claremont (AU)

Correspondence Address: MERCHANT & GOULD PC, P.O. BOX 2903, MINNEAPOLIS, MN, 55402-0903, US

(73) Assignee: SOLBEC PHARMACEUTICALS CO., LTD., Nedlands (AU)

(21) Appl. No.: 10/758,939

(22) Filed: Jan. 15, 2004

Related U.S. Application Data
(60) Provisional application No. 60/441,060, filed on Jan. 15, 2003.

(51) Int. Cl. A61K 31/7052; A61K 31/58

(52) U.S. Cl. 514/26; 514/173; 514/176

(57) ABSTRACT

Use of a glycoalkaloid composition containing at least one Z Glycoalkaloid of formula 1: 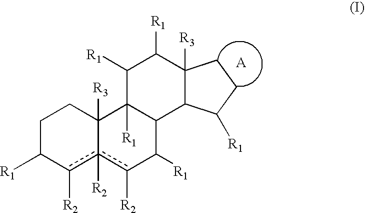 wherein: either one or both of the dotted lines represents a double bond, and the other a single bond, or both represent single bonds;
A: represents a radical selected from the following radicals of general formulae (II) to (V): 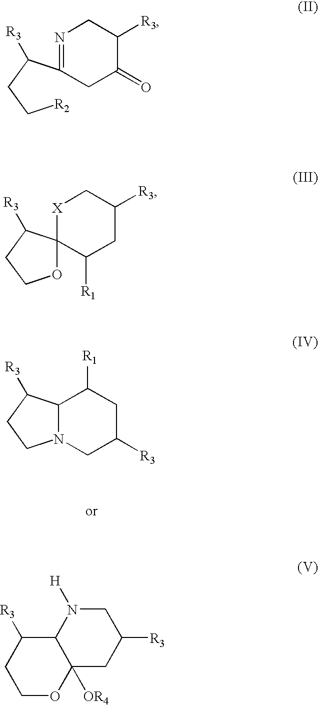
each of R¹ is a radical separately selected from the group consisting of hydrogen, amino, oxo and OR⁴;
each of R² is a radical separately selected from the group consisting of hydrogen, amino and OR⁴;
each of R³ is a radical separately selected from the group consisting of hydrogen, carbohydrate and a carbohydrate derivative;
“X” is a radical selected from the group comprising —CH₂—, —O— and —NH₂—; and
wherein the compound includes at least one R⁴ group that is a carbohydrate or a derivative such as one selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates; as an IL-6 antagonist.

### FIELD OF THE INVENTION

The present invention relates to methods of modulating IL-6 levels through the use of agents that have been found to have an affect on IL-6 production. The present invention also relates to the use of these agents to treat IL-6 related diseases and disorders, to modulate bone metabolism, to manipulate the immune system and in other applications for which their IL-6 modulating activity renders them useful such as screening methods. More particularly, the present invention relates to a method of killing cells, such as IL-6 producing cells, without causing an increase in IL-6 levels.

### BACKGROUND

IL-6 is a multifunctional cytokine produced by a number of different cell types. It achieves its effects through binding the ligand specific IL-6 receptor and, unlike most other cytokine receptors, the IL-6 receptor is active in both membrane bound and soluble forms.

IL-6 has various physiological effects and a role in many diseases including cancer, autoimmune and inflammatory diseases and infectious diseases such as viral and bacterial infections. The broad range of effects and numerous disease roles of IL-6 make IL-6 inhibitors good therapeutic candidates and the development of such inhibitors continues to be a popular area of research.

With particular regard to cancer, increased IL-6 secretion is an undesirable by-product of many chemotherapy based cancer treatments. The increased IL-6 levels can cause undesirable side effects and may promote the growth and proliferation of cancer cells that survive the chemotherapy that has obvious negative implications.

BEC is a therapeutic agent with anti-cancer activity comprising a mixture of the triglycosides: solasonine and solamargine, their corresponding mono and diglycosides, solasodine (an aglycone) and free sugars. The applicant has now discovered properties of the glycosides in BEC that render them useful for modulating IL-6 secretion and identify them as candidates for treating IL-6 mediated diseases. Thus, the present invention seeks to provide improved methods for modulating IL-6 production and methods for treating IL-6 mediated diseases based on these newly recognised properties of the glycosides in BEC.

### SUMMARY OF THE INVENTION

The present invention provides a method of reducing IL-6 production comprising contacting an IL-6 producing cell with an effective amount of a glycoalkaloid composition.

The present invention also provides a method of disrupting the binding of IL-6 to its receptor comprising contacting the receptor or IL-6 with an effective amount of a glycoalkaloid composition. In a related aspect, the present invention provides a method of reducing the proliferation of IL-6 producing cells comprising contacting said cells with an effective amount of a glycoalkaloid composition.

The IL-6 modulating activity of the compositions herein render them useful in various applications. Thus, the present invention also provides methods for treating IL-6 related diseases and disorders. They can be used as primary therapy or as part of a combination or adjunct therapy.

The present invention also provides methods of modulating bone metabolism and treating diseases or disorders associated with bone metabolism as well as the use of the glycoalkaloid compositions herein for modulating or otherwise affecting B cell differentiation, proliferation of thymic and peripheral T cells, induction of T cell differentiation to cytolytic T cells, natural killer cell activation.

The glycoalkaloid compositions may also be used to reduce tumour cell aggressiveness, metastasis, invasiveness, tumour angiogenesis via a method comprising contacting the cancer with an effective amount of a glycoalkaloid composition.

The present invention also provides a method for modulating or otherwise affecting skin proliferation, megakaryocytopoiesis, macrophage differentiation, neural cell differentiation and proliferation, cachexia, endometriosis, menses or spermatogenesis.

Finally, the invention provides for the use of the glycoalkaloid compositions herein as IL-6 antagonists and agonists and methods of screening for the same.

### BRIEF DESCRIPTION OF THE DRAWINGS

FIG. 1 is a series of bar charts depicting the IC50 values (average of three separate experiments) for (A) a glycoalkaloid composition, (B) gemcitabine and (C) docetaxol in mesothelioma and control cell lines;

FIG. 2 is a line graph illustrating the toxicity of a glycoalkaloid composition on two control cell lines measured using the MTT assay;

FIG. 3 is a series of line graphs comparing the toxicity of (A) a glycoalkaloid composition, (B) gemcitabine and (C) docetaxol to five mesothelioma cell lines and two control cell lines [Legend: square=A549; diamond=JU77; circle=LO68; triangle=NO36; crossed square=ONE58; crossed diamond=STY51; and crossed circle=HT29]

FIG. 4 is a bar chart illustrating IL-6 production in a range of mesothelioma and control cell lines;

FIG. 5 is series of line graphs comparing IL-6 concentration and cell viability in the mesothelioma cell line JU77 treated with (A) a glycoalkaloid composition, (B) gemcitabine and (C) docetaxol [Legend: square=pg/mL IL-6; circle=% viable cells; and arrow=IL-6 concentration in the absence of test drug];

FIG. 6 is series of line graphs comparing IL-6 concentration and cell viability in the mesothelioma cell line ONE58 treated with (A) a glycoalkaloid composition, (B) gemcitabine and (C) docetaxol [Legend: square=pg/mL IL-6; circle=% viable cells; and arrow=IL-6 concentration in the absence of test drug];

FIG. 7 is series of line graphs comparing IL-6 concentration and cell viability in the mesothelioma cell line LO68 treated with (A) a glycoalkaloid composition, (B) gemcitabine and (C) docetaxol [Legend: square=pg/mL IL-6; circle=% viable cells; and arrow=IL-6 concentration in the absence of test drug];

FIG. 8 is series of line graphs comparing IL-6 concentration and cell viability in the mesothelioma cell line N036 treated with (A) a glycoalkaloid composition, (B) gemcitabine and (C) docetaxol [Legend: square=pg/mL IL-6; circle=% viable cells; and arrow=IL-6 concentration in the absence of test drug];

FIG. 9 is series of line graphs comparing IL-6 concentration and cell viability in the mesothelioma cell line STY51 treated with (A) a glycoalkaloid composition, (B) gemcitabine and (C) docetaxol [Legend: square=pg/mL IL-6; circle=% viable cells; and arrow=IL-6 concentration in the absence of test drug]; and

FIG. 10 is series of line graphs comparing IL-6 concentration and cell viability in the control cell line A549 treated with (A) a glycoalkaloid composition, (B) gemcitabine and (C) docetaxol [Legend: square=pg/mL IL-6; circle=% viable cells; and arrow=IL-6 concentration in the absence of test drug].

### DISCLOSURE OF THE INVENTION

Methods of Modulating IL-6 Production

The present invention provides a method of reducing IL-6 production comprising contacting an IL-6 producing cell with an effective amount of a glycoalkaloid composition.

The present invention is based on the surprising discovery that the glycoalkaloid compositions described herein have the ability to modulate IL-6 production in a range of cancer cell lines. In particular, the glycoalkaloid compositions were able to be used to kill cancer cells without causing a concomitant increase in IL-6 levels. Whilst the glycoalkaloids compositions specific mode of action is unknown, the glycoalkaloids may bind to or in some other way affect IL-6 or its receptor such that the ability of IL-6 to bind its receptor is reduced. Alternatively or in addition, the rapid necrotic action of the glycoalkaloids on the cells may cause the death of the cells without inducing the same signalling pathways associated with apoptopic cell death and thus does not activate the IL-6 pathway. In this regard, it has been observed that at sub-lethal doses the glycoalkaloids cause decreased IL-6 production in IL-6 producing cells.

The present invention also provides a method of disrupting the binding of IL-6 to its receptor comprising contacting the receptor or IL-6 with an effective amount of a glycoalkaloid composition.

The binding of IL-6 to its receptor causes cell proliferation. Thus, the present invention also provides a method of reducing the proliferation of IL-6 producing cells comprising contacting said cells with an effective amount of a glycoalkaloid composition.

For the purposes of the present invention the phrase “IL-6 producing cell” includes all cells that are capable of secreting IL-6 such as: stimulated monocytes, fibroblasts, and endothelial cells, macrophages, T-cells and B-lymphocytes, granulocytes, smooth muscle cells, eosinophils, chondrocytes, osteoblasts, mast cells, glial cells, and keratinocytes, glioblastoma cells and cancer cells including melanoma, squamous cell carcinoma, mesothelioma, basal cell carcinoma, ovarian, hepatic cancer, renal cell carcinoma, gastric cancer, Kaposi's carcinoma, neuroblastioma, prostate carcinoma, non small cell lung cancer, lymphoma, testicular cancer, leukemia, cervical cancer, multiple myeloma, osteo carcinoma, glioblastoma and colorectal cancer.

The glycoalkaloid composition used in the methods of the present invention may be BEC or other compositions that have at least one glycoalkaloid of formula I: 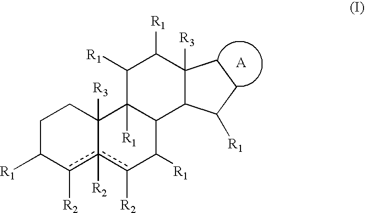
wherein: either one or both of the dotted lines represents a double bond, and the other a single bond, or both represent single bonds;
A: represents a radical selected from the following radicals of general formulae (II) to (V): 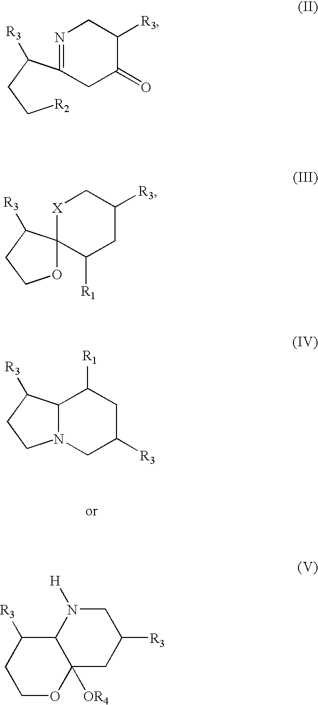
each of R¹ is a radical separately selected from the group consisting of hydrogen, amino, oxo and OR⁴;
each of R² is a radical separately selected from the group consisting of hydrogen, amino and OR⁴;
each of R³ is a radical separately selected from the group consisting of hydrogen, carbohydrate and a carbohydrate derivative;
“X” is a radical selected from the group comprising —CH₂—, —O— and —NH₂—; and
wherein the compound includes at least one R⁴ group that is a carbohydrate or a derivative such as one selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates. Glycoalkaloid compounds of this general formula are hereinafter referred to as “Z Glycoalkaloids”.

Preferably, the glycoalkaloid composition comprises at least two glycoalkaloids. Even more preferably, the composition is essentially without free sugars of the type that inhibit the IL-6 related activity of the glycoalkaloids therein.

When there are two glycoalkaloids in the composition, the ratio of the glycoalkaloids may be between about 6:1 and 1:6, about 1:4 and 4:1, about 1:3 and 3:1 or about 1:2 to 2:1. Alternatively, they may be present in a ratio selected from the group of ratios consisting of approximately: 1:6-1:0.5; 1:5; 1:4; 1:3; 1:2, 1:1.5 and 1:1. In this regard, compositions containing particular ratios of glycoalkaloids may have potentiated activity relative to (i) compositions containing the individual glycoalkaloids and (ii) BEC.

When the glycoalkaloids in the composition are solamargine and solasonine in a 1:1 ratio the solamargine and solasonine may be isolated in that they are essentially free of (i) mono and diglycosides and, preferably, essentially free of (ii) free sugars such as mono, di, tri, oligo or polysaccharides and (iii) aglycone. However, unless steps are taken to stabilise the glycoalkaloids, it will be appreciated that even in an isolated glycoalkaloid composition of the present invention there will be a small amount of free sugars and mono and diglycosides that result from degradation of the glycoalkaloids.

Preferably, the glycoalkaloids in the glycoalkaloid composition used in the method of the present invention are triglycoside glycoalkaloids, solasodine glycosides or are selected from the group of glycoalkaloids consisting of: solamargine, solasonine, solanine, tomatine, solanocapsine and 26-aminofurostane.

The glycoalkaloids in the glycoalkaloid composition used in the methods of the present invention may be chiral, steroisomers and mixtures thereof including enantiomers and/or diastereoisomers. Furthermore, the glycoalkaloids in the glycoalkaloid composition used in the methods of the present invention may be isolated from natural sources, synthesized or produced by chemically modifying other glycoalkaloids.

Preferably, the glycoalkaloids in the glycoalkaloid composition used in the methods of the present invention are triglycoside alkaloids and constitute greater than 70%-90% of the glycosides in the composition, more preferably 91-95% and even more preferably 96-100% of the glycosides in the glycoalkaloid composition.

The IL-6 modulating activity of the compositions herein render them useful in various applications, a number of which are discussed hereunder.

Methods of Invoking Cell Death

The ability of the compounds of the present invention to kill cells without a concomitant increase in IL-6 levels renders them useful as agents for killing or otherwise reducing cell viability. Thus, the present invention also provides a method for reducing the cell viability comprising the step of contacting said cell with an effective amount of a glycoalkaloid composition.

The cells may be varied and include IL-6 secreting cells and cells that are activated by IL-6. Preferably, the cells are diseased or otherwise undesirable.

Preferably, the cell viability is reduced by killing the cell. However, cell viability may be reduced by retarding cell proliferation or otherwise affecting the cell in a manner that does not actually kill the cell.

The cell killing ability of the glycoalkaloids compositions also render them useful for targeted killing of cells. Thus, the present invention also provides a glycoalkaloid composition comprising a cell targeting agent that delivers the glycoalkaloids to a predetermined cell or cell type. A related aspect of the invention is a method of killing a cell in a cell population in a targeted manner comprising contacting said population with a glycoalkaloid composition comprising a cell targeting agent adapted to deliver the glycoalkaloid to the target cell.

IL-6 Antagonists and Aqonists

The present invention also provides for the use of the glycoalkaloid compositions herein as IL-6 antagonists or agonists and methods of screening compounds to identify agents that enhance or block the binding of IL-6 to its receptor.

For example, IL-6 may be contacted with a labelled glycoalkaloid composition of the present invention in the absence or the presence of a candidate agonist or antagonist. The ability of the candidate molecule to bind IL-6 or the IL-6 receptor is reflected in decreased binding of the labelled glycoalkaloid composition. Molecules that bind gratuitously, i.e., without activating the IL-6 receptor and causing signal transduction are most likely to be good antagonists as these may be used to control IL-6 secretion and thus IL-6 related cell proliferation. Molecules that bind IL-6 well and prevent it from binding the receptor are also likely to be useful for controlling IL-6 secretion.

The effects of potential agonists and antagonists on IL-6 secretion may by measured, for instance, by contacting IL-6 producing cells with IL-6 concurrently or prior to contacting an IL-6 producing cell with the antagonist or agonist, and comparing the effect with that of cells contacted only with the IL-6.

Another example of an assay for antagonists is a competitive assay that combines an IL-6 receptor and a potential antagonist with IL-6 under appropriate conditions for a competitive inhibition assay. The IL-6 receptor can be labelled, such as by radioactivity, such that the binding of the receptor to IL-6 can be determined accurately to assess the effectiveness of the potential antagonist.

Potential antagonists include small organic molecules, peptides, polypeptides and antibodies that bind to IL-6 or the receptor at the same site as the glycoalkaloid compositions of the present invention and thus prevent the binding of the glycoalkaloid compositions. Potential antagonists also may be small organic molecules, a peptide, a polypeptide such as a closely related protein or antibody that binds to an alternative site on the IL-6 or receptor and prevents the action of the glycoalkaloid composition by excluding its binding to the IL-6 or its receptor.

IL-6 Related Diseases

The ability of the glycoalkaloid compositions herein to modulate IL-6 production renders them useful for treating IL-6 related diseases. Thus, the present invention also provides for the use of glycoalkaloid compositions to treat an IL-6 related disease.

The effective doses for the IL-6 related applications are preferably approximately 0.05%-1% glycoalkaloids, 0.05%-0.5% glycoalkaloids or 0.05-0.25% glycoalkaloids.

The IL-6 related disease may be varied and includes: inflammatory diseases such as rheumatoid arthritis; microbial diseases such as HIV, chronic fatigue syndrome and malaria; heart disease such as cardiac myopathy and cardiac disease progression; and other diseases such as Alzheimer's disease, arteriosclerosis, thyroiditis, Castleman's disease, paraneoplastic symptoms associated with cardiac myxoma, sepsis, psoriasis, diabetes, amyloidosis, hyperlipidemia, polycythemia vera, thrombocythemia and myocardial infarction.

The glycoalkaloid composition may be administered as the primary treatment agent or alternatively may be administered as an adjunct or as a component of a combination therapy. Thus, the present invention also provides for the use of a glycoalkaloid composition in combination with or as an adjunct to another agent for treating an IL-6 related disease.

The present invention also provides for the use of at least one Z Glycoalkaloid for preparing a medicament for treating an IL-6 related disease.

Bone Metabolism

IL-6 is involved in bone metabolism and thus the present invention also provides a method of modulating bone metabolism comprising contacting an osteoblast with an effective amount of a glycoalkaloid composition. These methods include the use of the glycoalkaloid compositions for induction of osteoclastogenesis and osteoclast activity.

The ability of the glycoalkaloid compositions to modulating bone metabolism renders them useful for treating diseases or disorders associated with bone metabolism. Thus, the present invention also provides a method of treating a bone related disease or disorder comprising administering an effective amount of a glycoalkaloid composition to a subject in need thereof.

The bone related disease or disorder may be varied and includes osteoporosis and osteoarthritis.

The present invention also provides for the use of at least one Z Glycoalkaloid for preparing a medicament for treating a bone metabolism related disease.

IL-6 Immunological/Inflammatory Roles

The IL-6 related activity of the glycoalkaloid compositions herein render the useful for modulating or otherwise affecting B cell differentiation, proliferation of thymic and peripheral T cells, induction of T cell differentiation to cytolytic T cells, natural killer cell activation.

The present invention also provides for the use of at least one Z Glycoalkaloid for preparing a medicament for treating a immune system related disease.

Cancer

IL-6 has a role in many cancers. Thus, the present invention also provides a method of treating cancer comprising administering to a subject an effective amount of a glycoalkaloid composition.

The cancers treated according to the present invention may be selected from the group comprising: melanoma, squamous cell carcinoma, mesothelioma, basal cell carcinoma, ovarian, hepatic cancer, renal cell carcinoma, gastric cancer, Kaposi's carcinoma, neuroblastioma, prostate carcinoma, non small cell lung cancer, lymphoma, testicular cancer, leukemia, cervical cancer, multiple myeloma, osteo carcinoma, glioblastoma and colorectal cancer.

IL-6 is involved in cancer cell proliferation and thus the present invention also provides a method of reducing the proliferation of cancer cells comprising contacting the cancer cell with an effective amount of a glycoalkaloid composition.

IL-6 also has a role in disease progression including the aggressiveness, invasiveness and metastasis of cancer. Thus, the present invention also provides a method of reducing tumour cell aggressiveness, metastasis, invasiveness, tumour angiogenesis comprising contacting the cancer with an effective amount of a glycoalkaloid composition.

Similarly, IL-6 promotes tumour angiogenesis. Thus, the present invention also provides a method of reducing tumour growth comprising contacting the tumour with an effective amount of a glycoalkaloid composition.

The effective amounts referred to herein may be determined by a person skilled in the art and preferably the glycoalkaloid compositions comprise about 0.001%-5% or 10% glycoalkaloids, more preferably 0.01%-5% or 10% and even more preferably 0.1%-5% or 10% glycoalkaloids. Put another way the glycoalkaloid compositions may contains about 1 to about 200 mg glycoalkaloid, about 100 ug-100 mg of glycoalkaloids, about 200 ug-50 mg of glycoalkaloids or 200 ug-10mg glycoalkaloids.

Other Applications

The glycoalkaloid compositions of the present invention may also be used to modulate or otherwise affect skin proliferation, megakaryocytopoiesis, macrophage differentiation, neural cell differentiation and proliferation, cachexia, endometriosis, menses or spermatogenesis. Thus, the present invention also extends to the use of at least one Z Glycoalkaloid for preparing a medicament for treating any one or more diseases resulting from abnormalities in these physiological activities.

The present invention will now be described with reference to the following Examples. The description of the Examples in no way limits the scope of the preceding description.

### EXAMPLES

### Example 1

### The Effects of Glycoalkaloids on Cell Growth and IL-6 Production

(a) Materials and Methods

(i) Cell lines

A panel of five human malignant mesothelioma cell lines, established from the pleural fluid of patients with the disease. These cell lines, designed Ju77, Lo68, No36, One58 and Sty51 have been previously described (1). None of the patients from whom the cell lines were derived had been exposed to chemotherapeutic agents. Two cell lines were purchased from ATCC (Rockville, Md.) for use as controls: HT29—a human colon adenocarcinoma (ATCC HTB38) and A549—a human lung adenocarcinoma (ATCC CCL185). These cell lines are representative of tumours that are generally drug resistant but have shown some responses to newer chemotherapy agents. They have previously been shown to have a similar degree of drug resistance to mesothelioma cell lines in vitro.

Cells were maintained in RPMI-1640 containing 5% fetal calf serum (Life Technologies Inc., Melbourne), 5×10⁻⁵ M 2-mercaptoethanol, 20 mM HEPES buffer, 100 IU benzylpenicillin/ml (CSL, Perth) and 50 μg gentamicin/ml (David Bull Laboratories, Melbourne), in water saturated air containing 5% CO₂ at 37° C. This growth medium will henceforth be called RF-5. All cells were checked at 3 monthly intervals for Mycoplasma and remained negative to testing.

(ii) Chemotherapy Agents

Docetaxel (Rhone-Poulenc Rorer, NSW) and gemcitabine (Eli Lilly Australia, NSW), vinorelbine (Pierre Fabre Oncology, Hampshire, England) were purchased. All drugs were stored as advised by the manufacturers, made up as necessary in recommended solvents as stock solutions and used within the period of stability for each agent as determined by the company. Dilutions of each drug were made fresh on the day of use in RF-5 from these stocks.

(iii) Glycoalkaloid Composition—SBP002 was a Sterile Aqueous Solution Containing 50mg of the Glycoalkaloids—Solamargine and Solasonine at a 1:1 Ratio in 1 mL of Acetic Acid (pH3-5).

(iv) MTT assay

Drug sensitivities for each cell line were determined by use of the MTT (Sigma-Aldrich, NSW) assay modified from the method described by Mossman (2). Cells were seeded in 100 μl of RF-5 at appropriate concentrations to maintain logarithmic growth for the duration of the assay in 96 well flat bottomed tissue culture plates. The plates were incubated for 24 hours to ensure stable growth and then dilutions of drug were added to each well in triplicates to make a total volume of 200 μl. Control wells were made for each plate with no drug or with no cells. Plates were then incubated for 72 hours after which 50 μl of MTT (2 mg/ml) was added and a further 4 hours incubation carried out. Plates were then centrifuged at 2000 rpm for 5 minutes after which all media was carefully aspirated. 100 μl of DMSO was added to solubilize the formazon crystals and each plate placed on a shaker for 30 minutes. Optical densities were determined using a SpectraMax 250 plate reader (Molecular Devices) at 570 nm. Plates were blanked on the cell-free medium containing wells that had been treated otherwise in an identical manner.

All experiments were repeated at least three times. The standard deviation for triplicate values for each experiment was always less then 10%. The IC50 for each cell line treated with a particular drug was calculated as being the concentration of drug that resulted in a 50% reduction in the optical density of treated cells compared to untreated cells.

(v) IL-6 ELISA

The IL-6 assay was performed using a human IL-6 ELISA kit (eBioscience, USA) according to the manufacturers protocol. Briefly ninety six well plates were incubated at room temperature overnight with 100 μL per well of monoclonal antihuman IL-6 antibody in coating buffer (0.1M Na₂CO₃/NaHCO₃, pH 9.6). The plate was washed five times with PBS and then 200 μL of blocking buffer added to each well and incubated for one hour at room temperature. The plate was then washed five times in PBS with 0.005% Tween 20 (Sigma-Aldrich, NSW). 100 μL of each sample to be assayed was added to each well and also IL-6 standard solutions ranging from 0.6-400 pg/ml were used to generate a standard curve.

After a one hour incubation, the plate was washed as above and 100 μL of biotinylated-IL-6 antibody solution added for a further hour. Following further washes, 100 μL of streptavidin-HRP conjugate solution was added for 30 minutes and the plates washed. 100 μL of ABTS solution was added and following incubation for 30 minutes at 37° C., the plate was read at 405 nm on a SpectraMax 250 plate reader (Molecular Devices) and the concentration of IL-6 for each sample calculated from the standard curve.

Samples for use in the ELISA were prepared by seeding of cells in 96 well plates with or without chemotherapy as described previously. As in the previous experiments chemotherapy was added 24 hours after cell seeding. For cell lysis experiments 300 μl of distilled water was added to each well and lysis of cells was confirmed by visual inspection. Media from these assays was removed at the appropriate time and stored at −20° C. until the ELISA was performed. The concentration of IL-6 for each cell line represents the mean value generated from three separate experiments. The results from chemotherapy effects on IL-6 production are representative of two or more experiments.

(b) Results

(i) In Vitro Drug Sensitivity in Mesothelioma

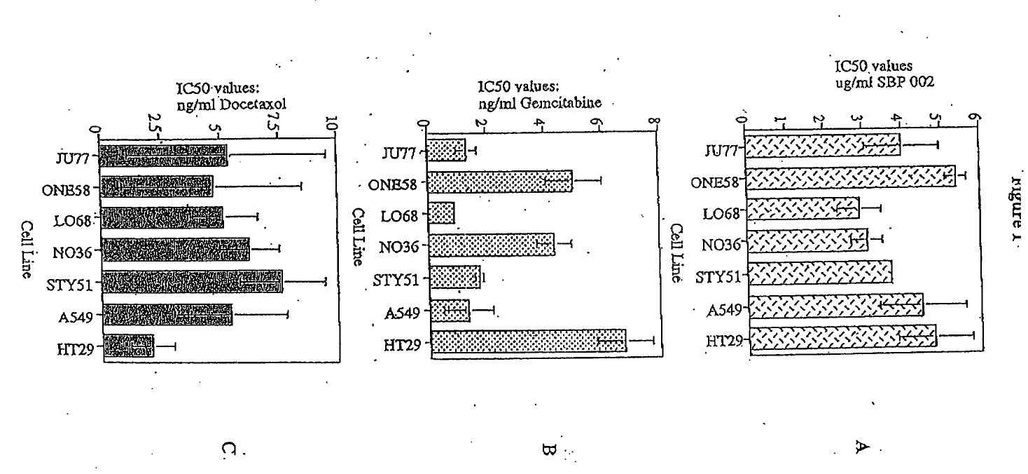

The concentration of each drug required to reduce cell viability by 50% (IC50) was calculated using the MTT assay. The IC 50 values for SBP 002 were calculated by using one fold increments in concentrations close to the IC 50 value. In the case of gemcitabine and docetaxol two fold increases of drug were used. The five mesothelioma cell lines had IC50s for SBP 002 ranging from 2.9 ug/ml to 5.4 ug/ml with LO68 being the most sensitive line and ONE58 the least sensitive line (FIG. 1A). The sensitivity of control cell lines A549 and HT29 to SBP 002 also fell within this range.

IC50 values of cells treated with gemcitabine ranged from 0.8 ng/ml to 5 ng/ml, a greater than five fold difference in sensitivity between the greatest and least sensitive cell (FIG. 1B). The IC50 for the lung cancer cell line, A549, fell within this range (1.75 ng/ml). The colon cancer cell line, HT29, had an IC50 higher than the other examined cell lines (6.7 ng/ml). For docetaxel, HT29 was the most sensitive (2 ng/ml) as reported previously and the five mesothelioma cell lines had IC50s ranging from 4.7 ng/ml to 7.6 ng/ml (FIG. 1C). The IC50 for A549, fell within this range at 5.4 ng/ml. These results for gemcitabine and docetaxol are all similar to those reported previously (3).

(ii) Drug Toxicity

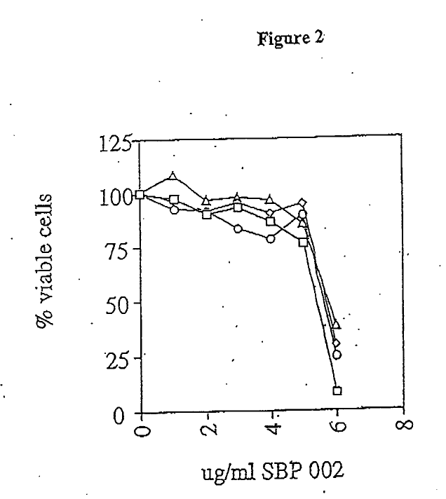

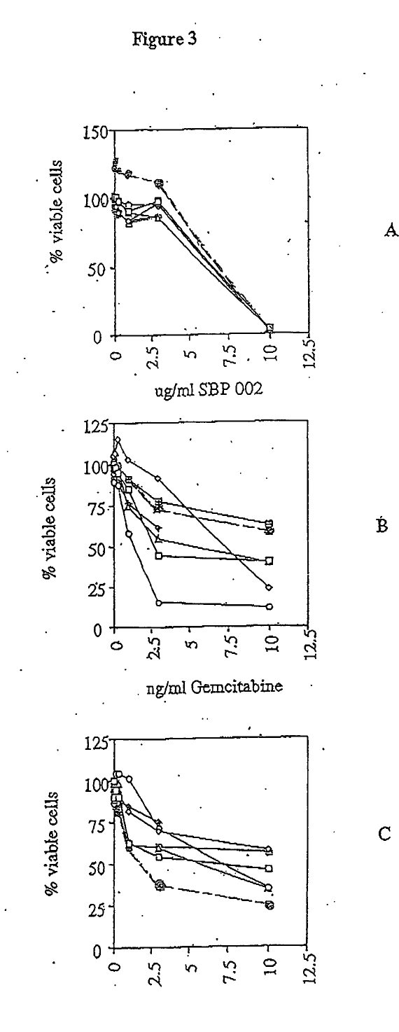

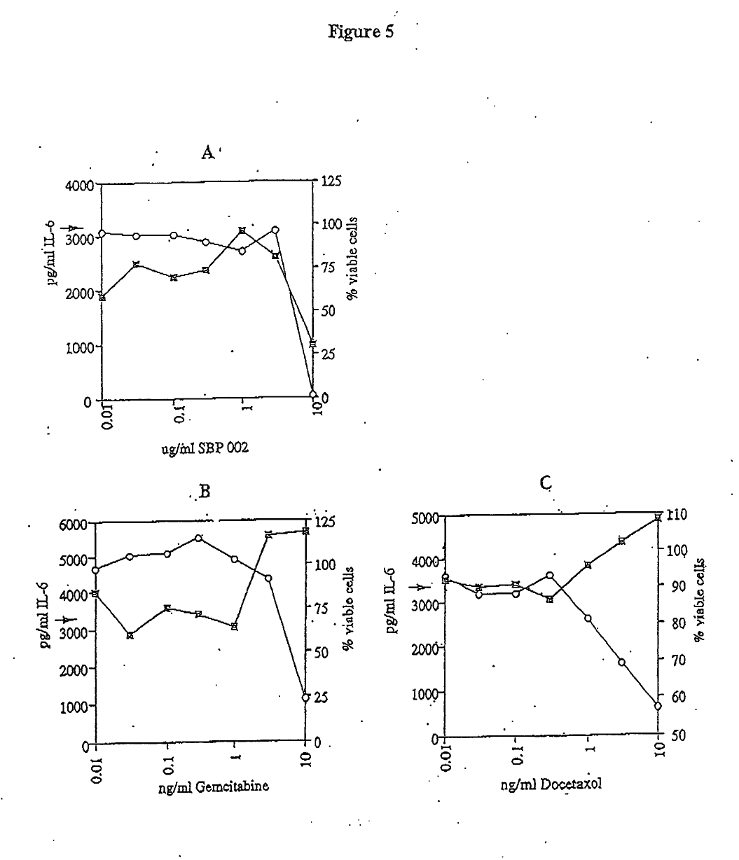

SBP 002 was toxic to both mesothelioma and control cells alike (FIG. 2). Viability remains high until a critical dose at around 5 ug/ml SBP 002 is reached. Thereafter viability falls sharply within a small dose increment. This contrasts gemcitabine and docetaxol where the dose response curve is linear over a much broader range (FIGS. 3B and 3C). For example, the death of NO36 cells treated with gemcitabine reaches a plateau at a concentration of approximately 3 ng/ml and at 10 ng/ml gemcitabine there is still 50% viability. In the case of docetaxol the plateau effect is clear for all cell lines (FIG. 5C). This suggests that SBP 002 is a more potent drug then those previously investigated by our research group (3).

(iii) IL6 Secretion From Mesothelioma Cells

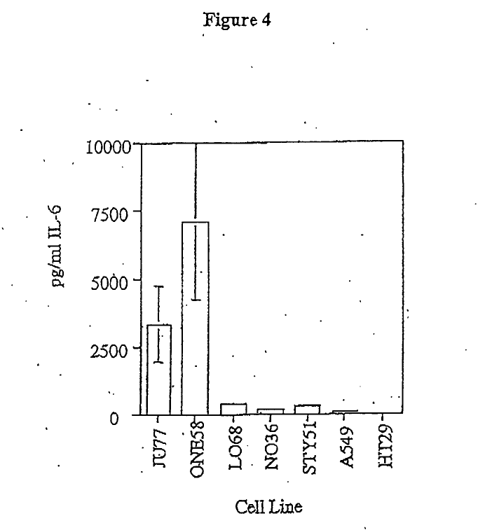

The five mesothelioma cell lines produced different amounts of IL-6 when grown in culture for 96 hours (FIG. 4). The greatest producers of IL-6 were ONE58 and JU77 while NO36 produced the least. The control cell line A549 produced minimal amounts of IL-6. However, no detectable IL-6 was secreted by HT29. The concentration of IL-6 for each cell line was determined from the final concentration present in the growth media following 96 hours of incubation. It should be noted that all cells were seeded at the same density, but because of variations in proliferation rates between the cell lines, the final cell numbers would have varied following 96 hours of growth. The doubling times for the five mesothelioma cell lines range from 25.3 hours to 33 hours. The cell lines ONE58 and NO36 were two of the slower growing cell lines and the difference in production of IL-6 between these cell lines and the others would have increased if total cell numbers were taken into account.

(iv) IL-6 Secretion from Mesothelioma Cells Treated with Chemotherapy

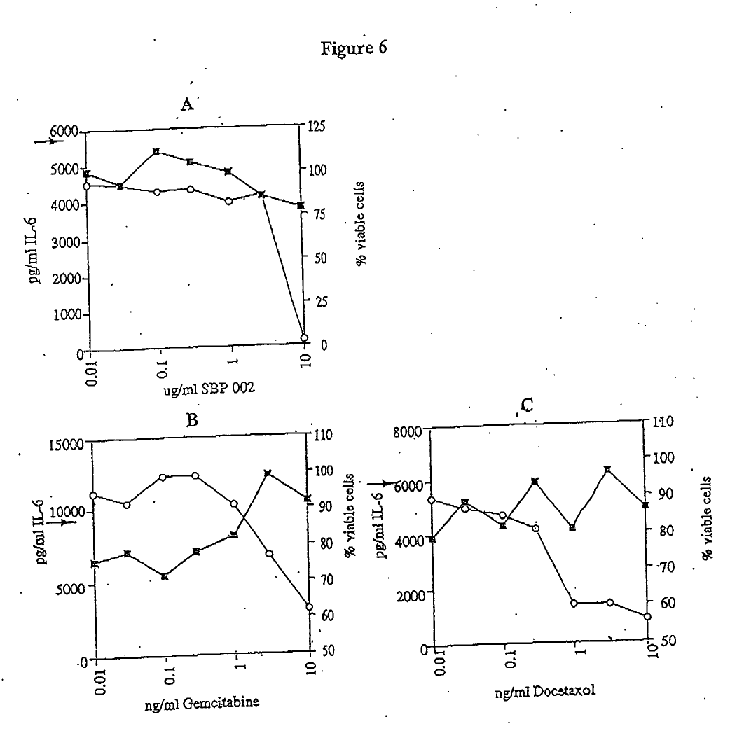

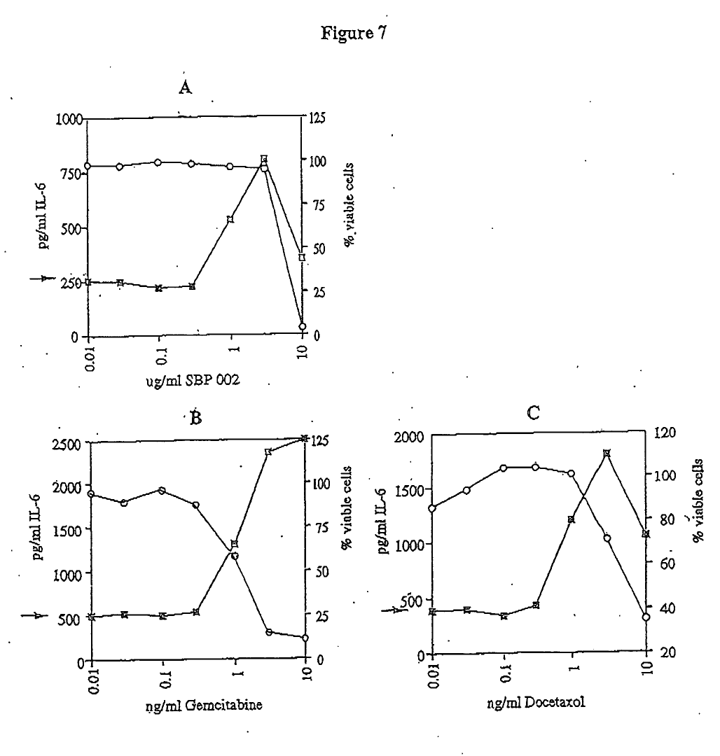

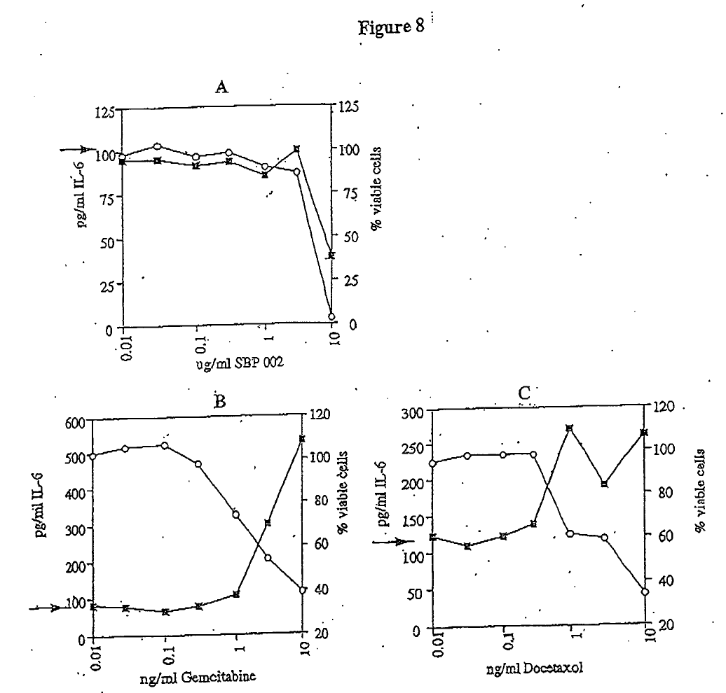

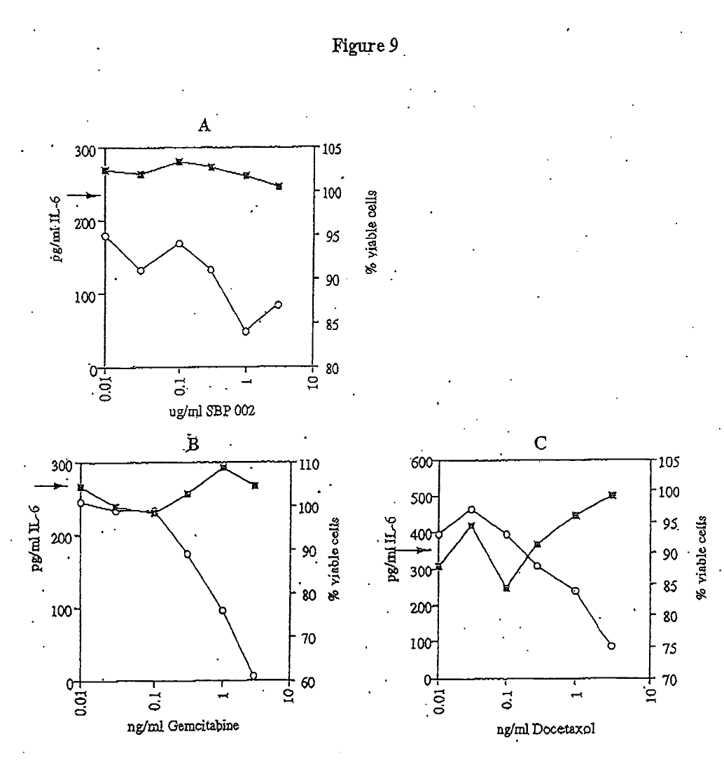

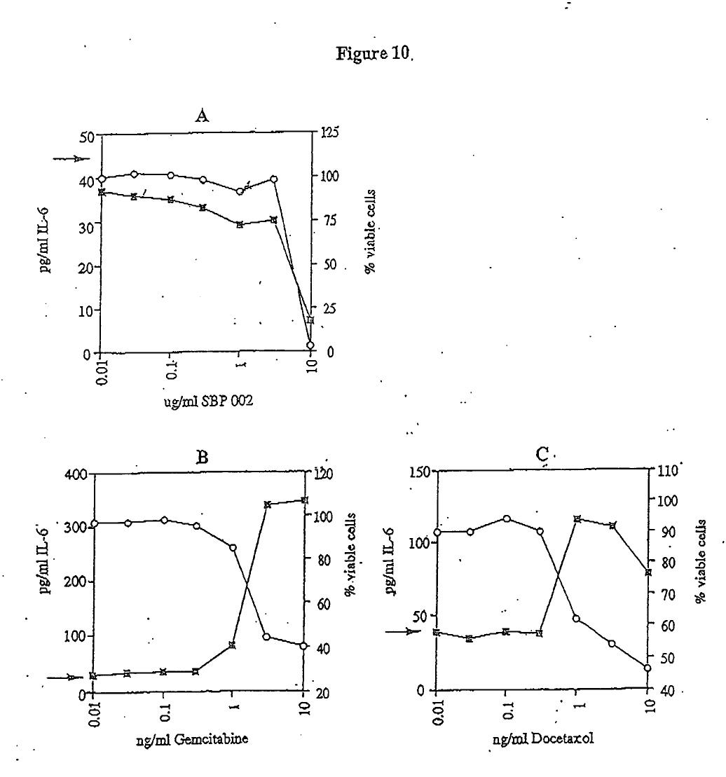

Mesothelioma and control cells treated with low concentrations of SBP 002 produced the same amount of IL-6 as untreated cells (FIGS. 5-10). IL-6 production remained constant at this level as the concentration of SBP 002 was increased. Thus SBP 002 did not mimic the effect reported for gemcitabine whereby treatment of mesothelioma cells with concentrations of gemcitabaine sufficient to produce a 5-30% reduction in relative cell growth, resulted in a decrease in the IL-6 concentration by up to one half of that released by untreated cells (3).

Treatment of cells with concentrations of docetaxol below its IC50 concentration did not effect IL-6 production compared to untreated cells. However, in the case of both gemcitabine and docetaxol a surge in IL-6 production was measured as the concentration of each drug approached its IC50 value (FIGS. 5-10). This surge of IL-6 did not occur when cells were treated with SBP 002. Instead, at concentrations above the IC 50 for SBP 002, IL-6 production decreased in proportion to the % loss of cell viability (FIGS. 5-10).

### Example 2

### Treatment of Psoriasis

(a) Materials/Methods

A 0.1% preparation of SBP002 in an aqueous cream base was applied daily to lesions on a subject suffering from psoriasis. This level represents a substantially lower dose than that applied to treat cancer.

(b) Results

Within 28 days the treated lesions had healed and been replaced by normal skin.

It was also noted that when the cream was applied to the calf or elbow lesions distal to the application point on the foot and hand also disappeared, evidencing systemic action.

Throughout the specification, unless the context requires otherwise, the word “comprise” or variations such as “comprises” or “comprising”, will be understood to imply the inclusion of a stated integer or group of integers but not the exclusion of any other integer or group of integers.

References

1. Manning L S, Whitaker D, Murch A R, Garlepp M J, Davis M R, Musk A W, Robinson B W: Establishment and characterization of five human malignant mesothelioma cell lines derived from pleural effusions. Int.J.Cancer 47:285-290, 1991

2. Mossman T (1983) Rapid colorimetric assay for cellular growth and survival: application to proliferation and cytotoxicity assays. Journal of Immunology Methods 65: 55-63

3. McClaren M J, Robinson B W S, Lake R A (2000) New chemotherapeutics in malignant mesothelioma: effects on cell growth and IL-6 production. Cancer Chemother Pharmacol 45: 502-508.

1. Use of a glycoalkaloid composition containing at least one containing at least one Z Glycoalkaloid of formula I: 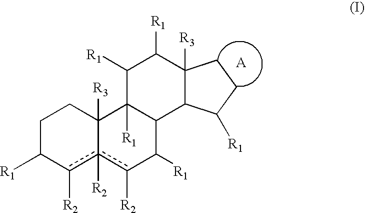
wherein: either one or both of the dotted lines represents a double bond, and the other a single bond, or both represent single bonds;
A: represents a radical selected from the following radicals of general formulae (II) to (V): 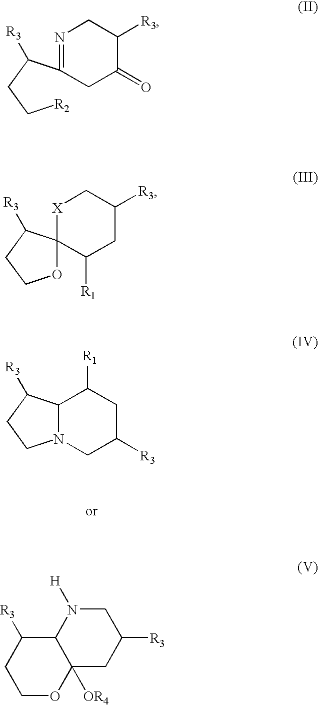
each of R¹ is a radical separately selected from the group consisting of hydrogen, amino, oxo and OR⁴;
each of R² is a radical separately selected from the group consisting of hydrogen, amino and OR⁴;
each of R³ is a radical separately selected from the group consisting of hydrogen, carbohydrate and a carbohydrate derivative;
“X” is a radical selected from the group comprising —CH₂—, —O— and —NH2—; and
wherein the compound includes at least one R⁴ group that is a carbohydrate or a derivative such as one selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates;
as an IL-6 antagonist.

2. A method for screening for IL-6 agonists or antagonists comprising the steps of contacting a glycoalkaloid composition containing at least one Z Glycoalkaloid with an IL-6 producing cell in the presence of a candidate agonist or antagonist and assessing IL-6 levels.

3. A method according to claim 2 wherein the Z Glycoalkaloid or the IL-6 receptor is labelled.

4. A method of reducing IL-6 production comprising contacting an IL-6 producing cell with an effective amount of a glycoalkaloid composition containing at least one Z Glycoalkaloid.

5. A method of disrupting the binding of IL-6 to its receptor comprising contacting the receptor or IL-6 with an effective amount of a glycoalkaloid composition containing at least one Z Glycoalkaloid.

6. A method of reducing the proliferation of IL-6 producing cells comprising contacting said cells with an effective amount of a glycoalkaloid composition containing at least one Z Glycoalkaloid.

7. A method according to claim 4 wherein the glycoalkaloid composition is essentially without free sugars of the type that inhibit the IL-6 related activity of the glycoalkaloids therein.

8. A method according to claim 4 wherein the Z Glycoalkaloids triglycoside glycoalkaloids, solasodine glycosides or are selected from the group of glycoalkaloids consisting of: solamargine, solasonine, solanine, tomatine, solanocapsine and 26-aminofurostane.

9. A method according to claim 4 wherein the glycoalkaloid composition comprises two Z Glycoalkaloids.

10. A method according to claim 9 wherein the ratio of the Z Glycoalkaloids is between about 6:1 and 1:6.

11. A method according to claim 9 wherein the ratio of the Z Glycoalkaloids is about 1:1.

12. A method according to claim 9 wherein the Z Glycoalkaloids are solamargine and solasonine in a 1:1 ratio and the solamargine and solasonine are essentially free of (i) mono and diglycosides.

13. A method according to claim 12 wherein the solamargine and solasonine are also essentially free of (i) free sugars such as mono, di, tri, oligo or polysaccharides and (ii) aglycone.

14. A method according to claim 4 wherein the Z Glycoalkaloids are chiral, stereoisomers and mixtures thereof including enantiomers and/or diastereoisomers.

15. A method according to claim 4 wherein the Z Glycoalkaloids are isolated from natural sources.

16. A method according to claim 4 wherein the Z Glycoalkaloids are triglycoside alkaloids and constitute greater than 70%-90% of the glycosides in the composition.

17. A method according to claim 4 wherein the Z Glycoalkaloids are triglycoside alkaloids and constitute 91-95% of the glycosides in the composition.

18. A method according to claim 4 wherein the Z Glycoalkaloids are triglycoside alkaloids and constitute 96-100% of the glycosides in the composition.

19. A method according to claim 4 wherein the glycoalkaloid composition is BEC.

20. The use of a glycoalkaloid composition containing at least one Z Glycoalkaloid to treat an IL-6 related disease.

21. The use according to claim 20 wherein the IL-6 related disease is selected from the group comprising: inflammatory diseases such as rheumatoid arthritis; microbial diseases such as HIV, chronic fatigue syndrome and malaria; heart disease such as cardiac myopathy and cardiac disease progression; and other diseases such as Alzheimer's disease, arteriosclerosis, thyroiditis, Castleman's disease, paraneoplastic symptoms associated with cardiac myxoma, sepsis, psoriasis, diabetes, amyloidosis, hyperlipidemia, polycythemia vera, thrombocythemia and myocardial infarction.

22. The use according to claim 20 wherein the glycoalkaloid composition is administered in combination with or as an adjunct to another agent for treating the IL-6 related disease.

23. A method of modulating bone metabolism comprising contacting an osteoblast with an effective amount of a glycoalkaloid composition containing at least one Z Glycoalkaloid

24. A method according to claim 23 wherein the glycoalkaloid composition induces osteoclastogenesis and/or osteoclast activity.

25. A method of treating a bone related disease or disorder comprising administering an effective amount of a glycoalkaloid composition containing at least one Z Glycoalkaloid to a subject in need thereof.

26. A method according to claim 25 wherein the bone related disease or disorder is osteoporosis or osteoarthritis.

27. Use of a glycoalkaloid composition containing at least one Z Glycoalkaloid to modulate or otherwise affect B cell differentiation, proliferation of thymic and peripheral T cells, induction of T cell differentiation to cytolytic T cells, natural killer cell activation.

28. A method of reducing the proliferation of cancer cells comprising contacting the cancer cell with an effective amount of a glycoalkaloid composition containing at least one Z Glycoalkaloid

29. A method of reducing tumour cell aggressiveness, metastasis, invasiveness, tumour angiogenesis comprising contacting the cancer with an effective amount of a glycoalkaloid composition containing at least one Z Glycoalkaloid.

30. A method of reducing tumour growth comprising contacting the tumour with an effective amount of a glycoalkaloid composition containing at least one Z Glycoalkaloid.

31. Use of a glycoalkaloid composition containing at least one Z Glycoalkaloid to modulate or otherwise affect one or more of skin proliferation, megakaryocytopoiesis, macrophage differentiation, neural cell differentiation and proliferation, cachexia, endometriosis, menses or spermatogenesis.

32. A method for reducing cell viability comprising the step of contacting said cell with an effective amount of a glycoalkaloid composition.

33. A method according to claim 32 wherein the cell is and IL-6 secreting cell or a cell that is activated by IL-6.

34. A method according to claim 32 wherein the cell is diseased or otherwise undesirable.

35. A method according to claim 32 wherein cell viability is reduced by killing the cell.

36. A method according to claim 32 wherein cell viability is reduced by retarding cell proliferation.

37. A glycoalkaloid composition comprising at least one Z Glycoalkaloid and a cell targeting agent that delivers the glycoalkaloid to a predetermined cell or cell type.

38. A method of killing a cell in a cell population in a targeted manner comprising contacting said population with a glycoalkaloid composition comprising at least on Z Glycoalkaloid and a cell targeting agent adapted to deliver the glycoalkaloid to the target cell.

39. A method according to claim 5 wherein the glycoalkaloid composition is essentially without free sugars of the type that inhibit the IL-6 related activity of the glycoalkaloids therein.

40. A method according to claim 6 wherein the glycoalkaloid composition is essentially without free sugars of the type that inhibit the IL-6 related activity of the glycoalkaloids therein.

41. A method according to claim 5 wherein the Z Glycoalkaloids triglycoside glycoalkaloids, solasodine glycosides or are selected from the group of glycoalkaloids consisting of: solamargine, solasonine, solanine, tomatine, solanocapsine and 26-aminofurostane.

42. A method according to claim 6 wherein the Z Glycoalkaloids triglycoside glycoalkaloids, solasodine glycosides or are selected from the group of glycoalkaloids consisting of: solamargine, solasonine, solanine, tomatine, solanocapsine and 26-aminofurostane.

43. A method according to claim 5 wherein the glycoalkaloid composition comprises two Z Glycoalkaloids.

44. A method according to claim 6 wherein the glycoalkaloid composition comprises two Z Glycoalkaloids.

45. A method according to claim 5 wherein the Z Glycoalkaloids are triglycoside alkaloids and constitute greater than 70%-90% of the glycosides in the composition.

46. A method according to claim 6 wherein the Z Glycoalkaloids are triglycoside alkaloids and constitute greater than 70%-90% of the glycosides in the composition.

47. A method according to claim 5 wherein the Z Glycoalkaloids are triglycoside alkaloids and constitute 91-95% of the glycosides in the composition.

48. A method according to claim 6 wherein the Z Glycoalkaloids are triglycoside alkaloids and constitute 91-95% of the glycosides in the composition.

49. A method according to claim 5 wherein the Z Glycoalkaloids are triglycoside alkaloids and constitute 96-100% of the glycosides in the composition.

50. A method according to claim 6 wherein the Z Glycoalkaloids are triglycoside alkaloids and constitute 96-100% of the glycosides in the composition.

51. A method according to claim 5 wherein the glycoalkaloid composition is BEC

52. A method according to claim 6 wherein the glycoalkaloid composition is BEC
